(19) United States
(12) Patent Application Publication
(10) Pub. No.: US 2022/0371018 A1
(43) Pub. Date: Nov. 24, 2022

(54) DROPLET COLLECTION UNIT, AND DROPLET COLLECTION APPARATUS AND METHOD

(71) Applicant: CANON MEDICAL SYSTEMS CORPORATION, Tochigi (JP)

(72) Inventor: Kenta KAMINO, Utsunomiya (JP)

(73) Assignee: CANON MEDICAL SYSTEMS CORPORATION, Tochigi (JP)

(21) Appl. No.: 17/663,923

(22) Filed: May 18, 2022

(30) Foreign Application Priority Data
May 18, 2021 (JP) 2021-083682
May 18, 2022 (JP) 2022-081343

(51) Int. Cl. B01L 3/00; C12Q 1/24; C12Q 1/70

(52) U.S. Cl. CPC B01L 3/502784; C12Q 1/24; C12Q 1/70; B01L 2200/061; B01L 2200/0673; B01L 2300/0636; B01L 2300/0864; B01L 2400/0424

(57) ABSTRACT

A droplet collection unit of an embodiment includes a generator and processing circuitry. The generator produces droplets each containing a microorganism and a substrate that reacts with an enzyme derived from the microorganism. The processing circuitry detects a reaction between the enzyme and the substrate in each droplet. The processing circuitry sorts the droplets on the basis of a detection result of the reaction.

### CROSS-REFERENCE TO RELATED APPLICATIONS

This application is based upon and claims the benefit of priority from Japanese Patent Application No. 2021-083682, filed on May 18, 2021, Japanese Patent Application No. 2022-081343, filed on May 18, 2022; the entire contents of which are incorporated herein by reference.

### FIELD

Embodiments disclosed herein and in the drawings relate to a droplet collection unit, and a droplet collection apparatus and method.

### BACKGROUND

In the related art, a method of producing a droplet in a microchannel to encapsulate a cell or a bacterium is known. For example, there are known techniques that use probability theory to encapsulate a cell or a bacterium within a droplet by one cell. When a cell is encapsulated in such a probabilistic manner, it is possible to fractionate only a droplet with an encapsulated cell by detecting the cell in the droplet with a microscope or the like.

### BRIEF DESCRIPTION OF THE DRAWINGS

FIG. 1 is a block diagram illustrating an example of the configuration of a droplet collection unit according to a first embodiment;

FIG. 2 is a diagram illustrating an example of a microfluidic chip according to the first embodiment;

FIG. 3 is a diagram for explaining an example of processing by the droplet collection unit according to the first embodiment;

FIG. 4 is a diagram for explaining an example of a system according to the first embodiment;

FIG. 5 is a diagram illustrating experimental results regarding a bacterium-derived enzyme (Q-galactosidase) according to the first embodiment; and

FIG. 6 is a diagram illustrating experimental results regarding a virus-derived enzyme (neuraminidase) according to the first embodiment.

### DETAILED DESCRIPTION

According to an embodiment, a droplet collection unit includes a generator and processing circuitry. The generator is configured to produce droplets each containing a microorganism and a substrate that reacts with an enzyme derived from the microorganism. The processing circuitry is configured to detect a reaction between the enzyme and the substrate in each droplet. The processing circuitry is configured to sort the droplets on the basis of a detection result of the reaction.

Embodiments of a droplet collection unit, and a droplet collection apparatus and method will be described in detail with reference to the accompanying drawings. Furthermore, the droplet collection unit and the droplet collection apparatus and method of the present application are not limited by the following embodiments. Furthermore, in the following description, similar components are given common reference numerals and duplicated explanations will be omitted.

### First Embodiment

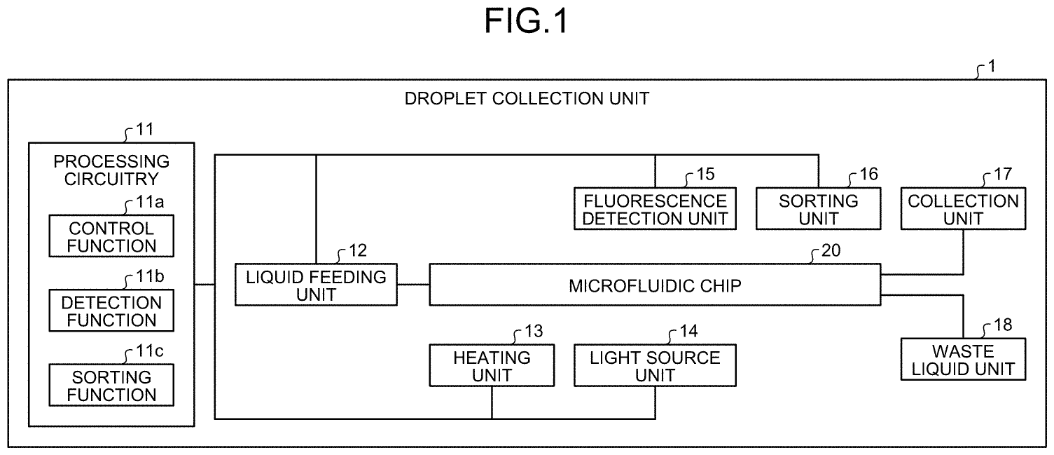

First, an example of the configuration of a droplet collection unit according to a first embodiment will be described. FIG. 1 is a block diagram illustrating an example of the configuration of a droplet collection unit 1 according to the first embodiment. The droplet collection unit 1 includes processing circuitry 11, a liquid feeding unit 12, a heating unit 13, a light source unit 14, a fluorescence detection unit 15, a sorting unit 16, a collection unit 17, a waste liquid unit 18, and a microfluidic chip 20, and collects droplets each containing a microorganism such as a virus and a bacterium.

The droplet collection unit 1 can be formed to be joinable to a system using a collected droplet. Specifically, the droplet collection unit 1 can be formed to be joinable to a system for various analyses and tests using a microorganism encapsulated in a droplet. For example, the droplet collection unit 1 can be formed to be joinable to a system for introducing various factors into a cell by using a virus encapsulated in a droplet as a vector.

The processing circuitry 11 controls various processes in the droplet collection unit 1. For example, the processing circuitry 11 is implemented by a processor. The processing circuitry 11 controls various processes in the droplet collection unit 1 in response to various operations via, for example, the droplet collection unit 1 or an input interface provided in the system described above.

The processing circuitry 11 reads and executes a computer program stored in storage circuitry (not illustrated) and performs a control function 11a, a detection function 11b, and a sorting function 11c. The processing circuitry 11 is an example of processing circuitry.

The control function 11a controls the delivery of liquid by the liquid feeding unit 12. Specifically, the control function 11a controls a pump or the like included in the liquid feeding unit 12 to send delivery liquid into the microfluidic chip 20. For example, the control function 11a controls the liquid feeding unit 12 to send various types of delivery liquids for producing droplets each containing a microorganism such as a virus and a bacterium into the microfluidic chip 20. Details of the delivery of the delivery liquid by the control function 11a will be described below.

Furthermore, the control function 11a adjusts the temperature of fluid in the microfluidic chip 20 by controlling the heating unit 13. Specifically, the control function 11a adjusts the temperature of the droplet in the microfluidic chip 20. Details of temperature adjustment by the control function 11a will be described below.

The detection function 11b detects a reaction in each droplet by controlling the light source unit 14 and the fluorescence detection unit 15. Specifically, the detection function 11b detects a reaction between an enzyme and a substrate in the droplet. More specifically, the detection function 11b detects a reaction in the droplet by emitting light by controlling the light source unit 14, irradiating the droplet produced in the microfluidic chip 20 with the light, and detecting fluorescence produced in the droplet by the fluorescence detection unit 15. For example, the detection function 11b detects fluorescence based on a reaction between an enzyme derived from a microorganism and a substrate, and detects a reaction between the enzyme and the substrate in the droplet by the detected fluorescence. Details of the detection process by the detection function 11b will be described below.

The sorting function 11c controls the sorting unit 16 to sort the droplets produced in the microfluidic chip 20. Specifically, the sorting function 11c sorts the droplets on the basis of the detection result by the detection function 11b. More specifically, the sorting function 11c determines the amount of microorganisms contained in each droplet on the basis of the detection result, and sorts the droplets on the basis of the determination result. For example, the sorting function 11c determines the number of microorganisms in each droplet on the basis of the detection result, and sorts the droplets on the basis of the determination result. Details of the droplet sorting process by the sorting function 11c will be described below.

The liquid feeding unit 12 sends the delivery fluid into the microfluidic chip 20 under the control of the control function 11a. Specifically, the liquid feeding unit 12 sends various types of delivery fluids for producing droplets each encapsulating a microorganism into the microfluidic chip 20. For example, the liquid feeding unit 12 includes a pump or the like, and sends various types of delivery fluids into the microfluidic chip 20. Details of the delivery liquid sent into the microfluidic chip 20 by the liquid feeding unit 12 will be described below.

The heating unit 13 adjusts the temperature of fluid in a channel in the microfluidic chip 20 under the control of the control function 11a. Specifically, the heating unit 13 adjusts the temperature of the droplets in the microfluidic chip 20. For example, the heating unit 13 is arranged in contact or non-contact with the microfluidic chip 20 and adjusts the temperature of the droplets in the microfluidic chip 20 by infrared heating, dielectric heating, and the like. The heating unit 13 may directly adjust the temperature of fluid, or adjust the temperature of fluid by adjusting the temperature of the microfluidic chip 20. Adjusting the temperature of a container may be regarded as adjusting the temperature of the droplets.

The heating unit 13 has a temperature sensor and can also be configured to measure the temperature of the droplets by the temperature sensor. In such a case, the control function 11a adjusts the heating by the heating unit 13 on the basis of the temperature measured by the temperature sensor in the heating unit 13. That is, the control function 11a can also control the temperature of the droplets in the microfluidic chip 20 to be maintained at a set temperature.

The light source unit 14 emits light under the control of the detection function 11b. Specifically, the light source unit 14 emits light to droplets flowing through the channel of the microfluidic chip 20. The light source unit 14 may be any one that can emit light capable of generating fluorescence from droplets, and is implemented by, for example, a laser or a light emitting diode (LED).

The fluorescence detection unit 15 is excited by the light emitted from the light source unit 14 and detects fluorescence produced from the droplets. The fluorescence detection unit 15 may be any one capable of detecting fluorescence emitted from the droplets, and is implemented by, for example, an image sensor, a fluorescence microscope, a fluorescence measuring instrument, and the like.

For example, the fluorescence detection unit 15 is arranged at a position facing the light source unit 14 with the microfluidic chip 20 interposed therebetween, detects fluorescence produced by excitation light emitted from the light source unit 14, and sends a detection result to the detection function 11b.

The sorting unit 16 sorts the droplets flowing through the microfluidic chip 20 under the control of the sorting function 11c. Specifically, the sorting unit 16 sorts the droplets according to the amount of microorganisms contained in each droplet. For example, the sorting unit 16 sorts droplets each containing a predetermined amount or more of microorganisms. Furthermore, for example, the sorting unit 16 sorts the droplets by the number of microorganisms contained in each droplet. For example, the sorting unit 16 includes an electrode for applying an electric field to the channel of the microfluidic chip 20, and sorts the droplets by dielectrophoresis by applying an electric field to the channel of the microfluidic chip 20 under the control of the sorting function 11c. As an example, the sorting unit 16 uses dielectrophoresis to sort droplets into the droplets each encapsulating a desired number of microorganisms and other droplets (the droplets encapsulating no microorganism and the droplets each encapsulating microorganisms the number of which is different from the desired number).

The collection unit 17 collects the droplets sorted by the sorting unit 16. Specifically, the collection unit 17 collects the droplets each encapsulating the desired number of microorganisms. The waste liquid unit 18 collects the other droplets sorted by the sorting unit 16.

The microfluidic chip 20 includes microchannels and produces droplets. Specifically, the microfluidic chip 20 produces droplets each encapsulating a microorganism and a substrate that reacts with an enzyme derived from the microorganism. Details of the microfluidic chip 20 will be described below. The microfluidic chip 20 is an example of a generator.

The above is a description of the configuration of the droplet collection unit 1 according to the present embodiment. Under such a configuration, the droplet collection unit 1 collects a droplet for which the number of microorganisms encapsulated in the droplet has been known. As described above, a probabilistic method is used to encapsulate a predetermined number of cells or the like in a droplet. For example, when the probabilistic method is used to produce a droplet encapsulating cells, a droplet encapsulating cells the number of which is greater (or less) than a predetermined number and a droplet encapsulating no cell (the number of encapsulated cells is zero) are also produced in addition to a droplet encapsulating a predetermined number of cells. Therefore, when a cell or the like is encapsulated in a droplet by the probabilistic method, the cell in the droplet is identified and sorted by a microscope or the like.

However, a small microorganism such as a virus is difficult to observe with an optical microscope, and it is not easy to identify whether microorganisms are encapsulated in a droplet, and identify the number of microorganisms when the microorganisms are encapsulated in the droplet.

In this regard, the droplet collection unit 1 of the present embodiment uses a reaction between an enzyme derived from a microorganism and a substrate to identify whether microorganisms are encapsulated in a droplet and identify the number of microorganisms when the microorganisms are encapsulated in the droplet, and collects a droplet for which the number of microorganisms encapsulated in the droplet has been known. Specifically, the droplet collection unit 1 probabilistically produces a droplet encapsulating a microorganism in the microfluidic chip 20, performs identification based on an enzyme reaction for each produced droplet, and sorts the droplets according to the identification result.

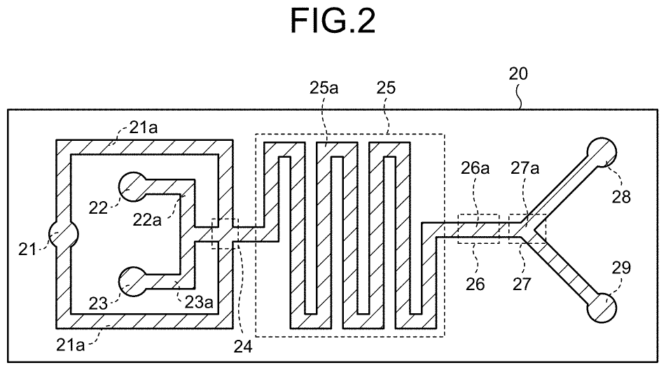

The following is a detailed description of the droplet collection unit 1 according to the present embodiment. FIG. 2 is a diagram illustrating an example of the microfluidic chip 20 according to the first embodiment. For example, the microfluidic chip 20 includes a first inlet 21, a second inlet 22, a third inlet 23, a droplet production area 24, an enzyme reaction area 25, a detection area 26, a sorting area 27, a collection port 28, and a waste liquid port 29. The droplet collection unit 1 can be configured so that the microfluidic chip 20 can be replaced each time a droplet collection method described below is performed.

The first inlet 21 is connected to a supply unit of first fluid for producing droplets, and the first fluid is injected by the delivery of the first fluid by the liquid feeding unit 12. The first fluid is continuous phase fluid for producing droplets. For example, the continuous phase fluid is oil, and oil corresponding to a microorganism to be encapsulated in a droplet is appropriately used. A channel 21a is formed between the first inlet 21 and the droplet production area 24, and the continuous phase fluid injected from the first inlet 21 flows in the direction to the droplet production area 24 by the delivery of the liquid feeding unit 12.

The second inlet 22 is connected to a supply unit of second fluid for producing droplets, and the second fluid is injected by the delivery of the second fluid by the liquid feeding unit 12. The second fluid is, for example, suspension of a microorganism, such as a virus, whose concentration is adjusted so that a predetermined number of microorganisms are encapsulated in a droplet on the basis of probability theory.

A target microorganism may be any microorganism that has an enzyme on the surface or inside of the microorganism and produces a product that can be optically detected by the reaction of the enzyme. For example, the target microorganism includes a virus of the family Paramyxoviridae with hemagglutinin-neuraminidase on the virus surface. The hemagglutinin neuraminidase reacts with a substrate “MUNANA (4-Methylumbelliferyl-N-acetyl-α-D-neuraminic acid)” to produce “4-methylumbelliferone” which is a fluorescent substance.

A channel 22a is formed between the second inlet 22 and the droplet production area 24, and the second fluid injected from the second inlet 22 flows in the direction to the droplet production area 24 by the delivery of the fluid by the liquid feeding unit 12.

The third inlet 23 is connected to a supply unit of third fluid for producing droplets, and the third fluid is injected by the delivery of the third fluid by the liquid feeding unit 12. The third fluid is, for example, a reagent containing a substrate that reacts with an enzyme present on the surface or inside of a microorganism. For example, the third fluid is a reagent containing the substrate “MUNANA”.

A channel 23a is formed between the third inlet 23 and the droplet production area 24, and the third fluid injected from the third inlet 23 flows in the direction to the droplet production area 24 by the delivery of the fluid from the liquid feeding unit 12.

In the microfluidic chip 20, the channel 22a and the channel 23a merge before the droplet production area 24, so that the second fluid flowing through the through channel 22a and the third fluid flowing through the channel 23a are mixed before the droplet production area 24, thereby forming dispersed phase fluid for producing droplets. That is, in the microfluidic chip 20, the dispersed phase fluid with a mixture of a microorganism and a substrate is formed in a confluence area of the channel 22a and the channel 23a.

The concentration of the microorganism in the second fluid and the concentration of the substrate in the third fluid are adjusted to be optimum concentrations when the second fluid is mixed with the third fluid to form the dispersed phase fluid. Furthermore, conditions such as PH in the dispersed phase fluid are appropriately adjusted according to a target enzyme.

The droplet production area 24 is an area where the channel 21a through which the continuous phase fluid flows intersects the channel through which the dispersed phase fluid flows (channel after the confluence area of the channel 22a and the channel 23a), and is connected in a state in which the channel 21a faces the channel through which the dispersed phase fluid flows. When the continuous phase fluid and the dispersed phase fluid are supplied to the droplet production area 24 by the liquid feeding unit 12, the continuous phase fluid shears the dispersed phase fluid and droplets are produced. For example, the droplet production area 24 produces droplets with a volume of femtoliter (fL) to picoliter (pL).

The droplet production area 24 continuously produces a large number of droplets as the continuous phase fluid and the dispersed phase fluid are sequentially sent by the liquid feeding unit 12. The droplets produced in the droplet production area 24 are sequentially sent to the enzyme reaction area 25 together with the continuous phase fluid as the first fluid to the third fluid are sent by the liquid feeding unit 12.

The enzyme reaction area 25 has a channel 25a for reacting an enzyme encapsulated in a droplet with a substrate and is connected to the droplet production area 24. In the channel 25a, the droplets produced in the droplet production area 24 are sequentially flowed together with the continuous phase fluid. The enzyme reaction area 25 is an area whose temperature is regulated by the heating unit 13. That is, the control function 11a regulates the temperature of the droplets flowing through the channel 25a in the enzyme reaction area 25 to the optimal temperature for an enzyme reaction by controlling the heating unit 13. At this time, when a microorganism is encapsulated in a droplet, an enzyme reacts with a substrate to produce a fluorescent substance. The channel 25a is formed with a length depending on the time required for a reaction between the enzyme and the substrate and a flow rate of the droplets.

The detection area 26 has a channel 26a to which light emitted from the light source unit 14 is emitted and, is connected to the enzyme reaction area 25. In the channel 26a, the droplets having flowed through the channel 25a in the enzyme reaction area 25 are sequentially flowed together with the continuous phase fluid. The channel 26a is made of a material that transmits the light emitted from the light source unit 14 and the fluorescence produced in the droplets, and is arranged between the light source unit 14 and the fluorescence detection unit 15.

In the detection area 26, light is emitted from the light source unit 14 to the channel 26a, and the fluorescence produced in each droplet is detected by the fluorescence detection unit 15. That is, in the detection area 26, light is emitted from the light source unit 14 to the droplet flowing through the channel 26a, and fluorescence is detected when microorganisms are encapsulated in the flowing droplet and a fluorescent substance is produced. The detection function 11b causes the light source unit 14 to emit light to the channel 26a and controls the fluorescence detection unit 15 to achieve detection information on fluorescence.

For example, the detection function 11b detects the number of microorganisms encapsulated in each droplet on the basis of the intensity of the fluorescence detected by the fluorescence detection unit 15. As an example, when no fluorescence is detected by the fluorescence detection unit 15, the detection function 11b detects that the number of microorganisms encapsulated in the droplet is “0”. Furthermore, the detection function 11b detects the number of microorganisms encapsulated in the droplet by comparing reference information indicating the relationship between the number of microorganisms encapsulated in a droplet and the fluorescence intensity with the intensity of the fluorescence detected by the fluorescence detection unit 15. The reference information indicating the relationship between the number of microorganisms encapsulated in a droplet and the fluorescence intensity is obtained in advance and stored in a storage circuitry (not illustrated).

The sorting area 27 has a branched flow channel 27a for sorting droplets and is connected to the detection area 26. In the flow channel 27a, the droplets having flowed through the flow channel 26a in the detection area 26 are sequentially flowed together with the continuous phase fluid. In the sorting area 27, the droplets are sorted by the sorting unit 16. That is, in the sorting area 27, droplets are sorted into the droplets each encapsulating a predetermined number of microorganisms and the other droplets by the operation of the sorting unit 16 under the control of the sorting function 11c.

Specifically, the sorting function 11c determines the number of microorganisms contained in each droplet on the basis of the intensity of fluorescence based on a reaction between the enzyme and the substrate, and sorts the droplets on the basis of the determination result. More specifically, the sorting function 11c controls the sorting unit 16 on the basis of the detection result detected by the detection function 11b to sort droplets into the droplet each encapsulating the predetermined number of microorganisms and the other droplets. For example, on the basis of a flow rate of the droplets (liquid feeding speed by the liquid feeding unit 12) and a distance between the detection area 26 and the sorting area 27, the sorting function 11c specifies the passage timing in the sorting area 27 of a droplet from which fluorescence corresponding to a predetermined number of microorganisms encapsulated in the droplet is detected. As an example, the sorting function 11c specifies the passage timing in the sorting area 27 of a droplet from which a fluorescence intensity indicating that the number of microorganisms encapsulated in the droplet is “1” is detected.

Then, the sorting function 11c causes the sorting unit 16 to apply an electric field at the specified passage timing, thereby moving a droplet in which the number of viruses encapsulated in the droplet is the predetermined number toward the collection port 28 and allowing the droplet to flow through the channel connected to the collection port 28. For example, the sorting function 11c moves the droplet in which the number of microorganisms encapsulated in the droplet is “1”, toward the collection port 28, and allows the droplet to flow to the collection port 28. The sorting area 27 is configured so that the droplets flow through a channel connected to the waste liquid port 29 in a state in which no electric field is applied by the sorting unit 16.

The collection port 28 is connected to the collection unit 17 and allows a droplet in which the number of microorganisms encapsulated in the droplet is the predetermined number to flow to the collection unit 17. The waste liquid port 29 is connected to the waste liquid unit 18 and allows a droplet encapsulating microorganisms the number of which is different from the predetermined number to flow to the waste liquid unit 18.

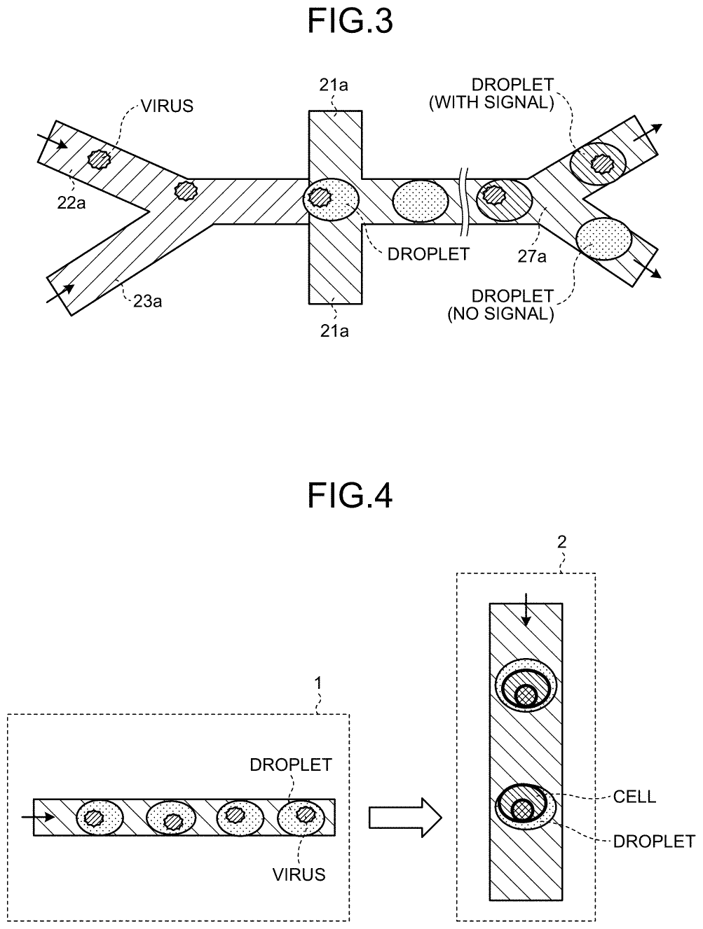

An example of processing by the droplet collection unit 1 is described below with reference to FIG. 3. FIG. 3 is a diagram for explaining an example of processing by the droplet collection unit 1 according to the first embodiment. FIG. 3 illustrates droplet production and droplet sorting in the microfluidic chip 20. Furthermore, FIG. 3 illustrates a case where a virus is targeted as a microorganism.

For example, as illustrated in FIG. 3, the second fluid containing a virus is supplied via the channel 22a and the third fluid containing a substrate is supplied via the channel 23a. Then, the second fluid and the third fluid are mixed in the confluence area of the channel 22a and the channel 23a to form the dispersed phase fluid. When the dispersed phase fluid reaches the droplet production area 24 intersecting with the channel 21a, the dispersed phase fluid is sheared by the continuous phase fluid supplied through the channel 21a and becomes a droplet.

Furthermore, when a droplet containing a virus is probabilistically generated, in addition to a droplet encapsulating a predetermined number of viruses, a droplet encapsulating no virus and a droplet encapsulating viruses the number of which is different from the predetermined number are produced.

The droplet collection unit 1 according to the present embodiment causes an enzyme derived from a virus to react with a substrate, detects a droplet in which the number of viruses encapsulated in the droplet is a predetermined number on the basis of fluorescence information obtained as a result of the reaction, and sorts droplets into the droplets in which the number of viruses encapsulated in each droplet is the predetermined number and the other droplets. That is, the droplet collection unit 1 collects droplets (with signal) each encapsulating a predetermined number of viruses and exhibiting fluorescence of a corresponding intensity to separate them from the other droplets (no signal) (droplets exhibiting no fluorescence and droplets exhibiting fluorescence different from the corresponding intensity). This enables the droplet collection unit 1 to collect a droplet for which the number of microorganisms encapsulated in the droplet has been known.

As described above, the droplet collection unit 1 according to the present embodiment produces droplets each containing a microorganism in the microfluidic chip 20, identifies the number of microorganisms encapsulated in each droplet on the basis of a reaction between an enzyme derived from the microorganisms and a substrate, and selectively collects a droplet encapsulating a predetermined number of microorganisms.

The microfluidic chip 20 in the droplet collection unit 1 is not limited to the shape illustrated in FIG. 2, but can be appropriately modified according to conditions such as droplet production and enzyme reactions. Furthermore, the channel width and channel length of each channel in the microfluidic chip 20 can also be appropriately modified according to the conditions such as droplet production and enzyme reactions.

The droplet collection unit 1 having the configuration described above can be formed to be joinable to a system using collected droplets. FIG. 4 is a diagram for explaining an example of a system according to the first embodiment. FIG. 4 illustrates the system including a droplet collection unit 1 that collects a droplet for which the number of viruses enclosed in the droplet has been known and a droplet collection unit 2 that collects a droplet encapsulating a cell.

In such a system, in order to introduce various factors into a cell by using a virus encapsulated in a droplet as a vector, a droplet encapsulating a virus is fused with a droplet encapsulating a cell. For example, as illustrated in FIG. 4, the above system fuses a droplet collected by the droplet collection unit 1 (droplet encapsulating one virus) with a droplet collected by the droplet collection unit 2 (droplet encapsulating one cell).

Although the droplet collection unit 1 according to the present embodiment uses a probabilistic method to produce a droplet encapsulating a virus, the droplet collection unit 1 can selectively separate a droplet for which the number of viruses encapsulated in the droplet (for example, one) has been known from droplets. Therefore, by using the droplet collection unit 1, the above system can reliably fuse a droplet encapsulating a virus with a droplet encapsulating a cell. The fusion of droplets can be performed efficiently. Furthermore, the droplet collection unit 1 can selectively separate a droplet for which the number of viruses encapsulated in the droplet has been known from droplets, thereby easily controlling the amount of virus infection to cells.

When droplets are simply produced by a probabilistic method, droplets encapsulating no virus and droplets encapsulating viruses the number of which is different from the desired number, in addition to droplets encapsulating viruses the number of which is the desired number (for example, one), are mixed in the produced droplets, resulting in inefficient fusion of droplets in the above system.

In the embodiment described above, the case where a virus of the family Paramyxoviridae with hemagglutinin-neuraminidase is targeted as a microorganism has been described. However, the embodiment is not limited thereto, and any microorganism having an enzyme on the surface or inside thereof and capable of producing a product that can be optically detected by the reaction of the enzyme may also be used. For example, examples of target microorganisms include viruses of the Coronaviridae family with hemagglutinin esterase, viruses such as a human immunodeficiency virus (HIV) with reverse transcriptase, and viruses such as an Ebola virus with RNA-dependent RNA polymerase. Furthermore, examples of the target microorganisms include bacteria such as coliform bacteria, Vibrio parahaemolyticus, Campylobacter, Enterobacter, and Bacillus bacteria.

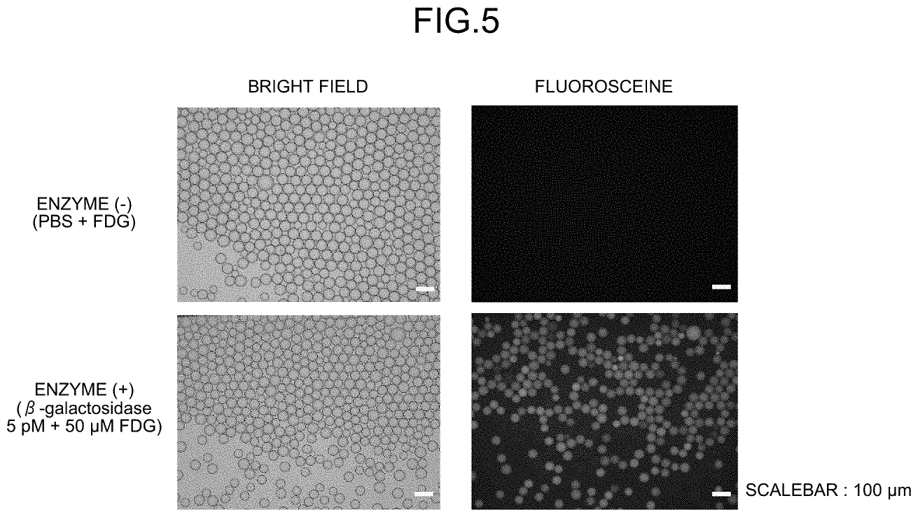

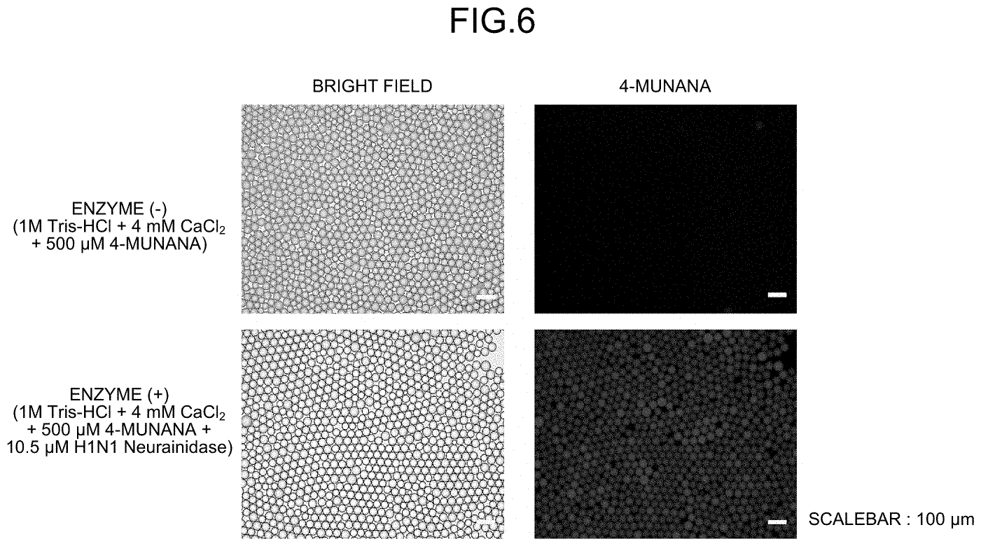

Hereinafter, the results of the experiment to detect enzyme reactions in droplets are described with reference to FIG. 5 and FIG. 6. FIG. 5 is a diagram illustrating experimental results regarding a bacterium-derived enzyme (R-galactosidase) according to the first embodiment. Furthermore, FIG. 6 is a diagram illustrating experimental results regarding a virus-derived enzyme (neuraminidase) according to the first embodiment.

### Experimental Example 1: Experiment on Bacterium-Derived Enzyme

### Reagent Used

Phosphate Buffered Saline (PBS) (−): manufactured by Nacalai Tesque Inc.

Fluorescein Di-β-D-galactopyranoside (FDG) (fluorescein): manufactured by Fujifilm Wako Pure Chemical Industries, Ltd.

β-galactosidase: FUJIFILM Wako Pure Chemical Industries, Ltd.

### Experimental Procedure

FDG was prepared by adding FDG to PBS (−) to a concentration of 100 μM. β-galactosidase was prepared by adding β-galactosidase to PBS (−) to a concentration of 10 pM. Fluorine-based surfactant dSURF (manufactured by Fluigent) was diluted with fluorine oil HFE7500 (3M) to prepare 0.5% dSURF. In a droplet production microchannel (manufactured by Chipshop), droplets were produced by pumping FDG solution and 10 pM β-galactosidase at 2 μL/min each from two dispersed phase inlets and pumping 0.5% dSURF at 20 μL/min from a continuous phase inlet. As a control experiment, droplets were produced by pumping only FDG solution and PBS (−) at 2 μL/min each from the two dispersed phase inlets and pumping 0.5% dSURF at 20 μL/min from the continuous phase inlet.

FIG. 5 illustrates the result of incubating the droplets produced by the aforementioned procedure at 37° C. for 1 hour and observing the droplets with a fluorescence microscope. In FIG. 5, the upper part indicates the result of the control experiment and the lower part indicates the result of the experiment on β-galactosidase. Furthermore, in FIG. 5, the left side indicates observed bright-field images of the droplets after the incubation in each experiment. Furthermore, the left side indicates observed images of the fluorescence of the droplets after the incubation in each experiment.

As shown in the bright-field images on the left side of FIG. 5, droplets were produced in each experiment. Furthermore, as shown in the lower image on the right side of FIG. 5, fluorescence in the droplets was observed in the presence of β-galactosidase. On the other hand, in the control experiment, as shown in the upper image on the right side of FIG. 5, no fluorescence was observed in the droplets. In this way, the enzyme reaction by β-galactosidase can be used to determine whether bacteria (for example, coliform bacteria or the like) are encapsulated in the droplets.

### Experimental Example 2: Experiment on Virus-Derived Enzyme

### Reagent Used

1M Tris-HCl pH 9.0: manufactured by Nippon gene Co., Ltd.

4-MUNANA: manufactured by Sigma

Neuraminidase, Influenza A Virus, H1N1, Recombinant, Carrier-free (neuraminidase): manufactured by R&D systems

### Experimental Procedure

4-MUNANA was prepared by adding 4-MUNANA to pH 9.0 1M Tris-HCl to a concentration of 1 mM. Neuraminidase was prepared by adding neuraminidase to pH 9.0 1M Tris-HCl to a concentration of 21 μM. Fluorine-based surfactant dSURF (manufactured by Fluigent) was diluted with fluorine oil HFE7500 (3M) to prepare 0.5% dSURF. In the droplet production microchannel (manufactured by Chipshop), droplets were produced by pumping 4-MUNANA solution and 21 μM neuraminidase at 2 μL/min each from the two dispersed phase inlets and pumping 0.5% dSURF at 30 μL/min from the continuous phase inlet. As a control experiment, droplets were produced by pumping only 4-MUNANA solution and 1M Tris-HCl at 2 μL/min each from the two dispersed phase inlets and pumping 0.5% dSURF at 30 μL/min from the continuous phase inlet.

FIG. 6 illustrates the result of incubating the droplets produced by the aforementioned procedure at 37° C. for 3 hours and observing the droplets with a fluorescence microscope. In FIG. 6, the upper part indicates the result of the control experiment and the lower part indicates the result of the experiment on neuraminidase. Furthermore, in FIG. 6, the left side indicates observed bright-field images of the droplets after the incubation in each experiment. Furthermore, the left side indicates observed images of the fluorescence of the droplets after the incubation in each experiment.

As shown in the bright-field images on the left side of FIG. 6, droplets were produced in each experiment. Furthermore, as shown in the lower image on the right side of FIG. 6, fluorescence in the droplets was observed in the presence of neuraminidase. On the other hand, in the control experiment, as shown in the upper image on the right side of FIG. 6, no fluorescence was observed in the droplets. In this way, the enzyme reaction by neuraminidase can be used to determine whether viruses are encapsulated in the droplets.

As described above, it is possible to determine whether a microorganism is encapsulated in a droplet by detecting fluorescence produced on the basis of an enzyme reaction. That is, the droplet collection unit 1 can sort droplets into droplets encapsulating microorganisms and droplets encapsulating no microorganism. Then, the droplet collection unit 1 sorts droplets by the number of microorganisms encapsulated in each droplet on the basis of the intensity of the detected fluorescence.

When a droplet encapsulating one microorganism is separated from droplets, fluorescence is weak and may be difficult to detect with a normal detection system. Therefore, in the droplet collection unit 1, a high-sensitivity camera can be applied as the fluorescence detection unit 15. Furthermore, the droplet collection unit 1 can increase detection sensitivity by slowing a flow rate of liquid sent from the inlet to increase an exposure time at an image sensor. In such a case, for example, the control function 11a controls the liquid feeding unit 12 to send delivery liquid at a delivery rate that is required for producing droplets and is also the slowest delivery rate. Furthermore, the detection function 11b controls a shutter in the fluorescence detection unit 15 so that the exposure time of the image sensor in the fluorescence detection unit 15 is as long as possible within an allowable range.

As described above, according to the first embodiment, the microfluidic chip 20 produces droplets each containing a microorganism and a substrate that reacts with an enzyme derived from the microorganism. The detection function 11b detects a reaction between the enzyme and the substrate in each droplet. The sorting function 11c sorts the droplets on the basis of the detection results of the reaction. Consequently, the droplet collection unit 1 according to the first embodiment can collect a droplet in which the amount of microorganisms encapsulated in the droplet is adjusted.

For example, a direct viral fluorescence method using fluorescently labeled antibodies is known as a method for detecting one virus. However, in the direct viral fluorescence method, since the virus is labeled using an antibody, the labeled substance affects various analyses and tests using the virus. On the other hand, the method according to the first embodiment can detect the number of viruses without labeling viruses, and does not affect various analyses and tests using viruses (for example, one-virus genome analysis, drug resistance tests, or the like).

Furthermore, for example, a method using a microwell is known as a method for detecting the presence or absence of viruses by enzymes derived from the viruses. However, in the method using a microwell, viruses are encapsulated in oil, making it difficult to collect viruses, and after viruses are detected, it is difficult to use the detected viruses for various analyses and tests. On the other hand, with the method according to the first embodiment, viruses can be isolated by droplets using microchannels and can be easily used for various analyses and tests.

Furthermore, according to the first embodiment, the sorting function 11c determines the amount of microorganisms contained in each droplet on the basis of a detection result of a reaction, and sorts the droplets on the basis of a result of the determination. Consequently, the droplet collection unit 1 according to the first embodiment enables sorting according to the amount of microorganisms encapsulated in each droplet.

Furthermore, according to the first embodiment, the sorting function 11c determines the number of microorganisms in each droplet on the basis of the detection result of the reaction, and sorts the droplets on the basis of the result of the determination. Consequently, the droplet collection unit 1 according to the first embodiment makes it possible to quantify the number of microorganisms encapsulated in each droplet.

Furthermore, according to the first embodiment, the detection function 11b detects fluorescence based on a reaction between an enzyme and a substrate. Consequently, the droplet collection unit 1 according to the first embodiment makes it possible to easily detect the presence or absence of microorganisms in each droplet.

Furthermore, according to the first embodiment, the sorting function 11c determines the number of microorganisms in each droplet on the basis of the intensity of fluorescence based on the reaction between the enzyme and the substrate, and sorts the droplets on the basis of the determination result. Consequently, the droplet collection unit 1 according to the first embodiment makes it possible to easily quantify the number of microorganisms in each droplet.

Furthermore, according to the first embodiment, the sorting function 11c sorts, by dielectrophoresis, the droplets produced in the microfluidic chip 20. Consequently, the droplet collection unit 1 according to the first embodiment enables easy sorting of the droplets in the microchannel.

Furthermore, according to the first embodiment, the control function 11a adjusts the temperature of the droplets. Consequently, the droplet collection unit 1 according to the first embodiment enables a reaction between the enzyme and the substrate to be carried out at an optimal temperature.

### Second Embodiment

In the first embodiment described above, the case where the presence or absence of a single type of microorganism is detected and droplets encapsulating the microorganism are sorted has been described. In the second embodiment, a case where multiple types of microorganisms are targeted and droplets encapsulating the microorganisms are sorted is described.

In the second embodiment, the second fluid injected from the second inlet 22 contains multiple species of microorganisms. The plurality of microorganisms contained in the second fluid have different enzymes. Furthermore, enzymes react with different substrates. The concentration of microorganisms in the second fluid is adjusted so that a predetermined number of (for example, one) microorganisms are probabilistically encapsulated in each droplet when the second fluid is mixed with the third fluid to form the dispersed phase fluid, regardless of the type of microorganisms.

Furthermore, in the second embodiment, the third fluid injected from the third inlet 23 contains a plurality of types of substrates that react with respective enzymes of the microorganisms contained in the second fluid. The concentration of each substrate in the third fluid is adjusted to an optimal concentration when the third fluid is mixed with the second fluid to form the dispersed phase fluid.

The liquid feeding unit 12 sends the second fluid and the third fluid described above to the microfluidic chip 20, as in the first embodiment. The second fluid and the third fluid sent to the microfluidic chip 20 are mixed to form the dispersed phase fluid. the dispersed phase fluid is sheared by the continuous phase fluid in the droplet production area 24, thereby forming droplets.

The droplets produced in the second embodiment include droplets containing only a plurality of substrates without microorganisms, droplets containing the substrates and one of the microorganisms, and droplets containing the microorganisms and the substrates.

The detection function 11b according to the second embodiment detects whether fluorescence is detected by the fluorescence detection unit 15 and the wavelength (or spectrum) and intensity of the fluorescence detected by the fluorescence detection unit 15. That is, the detection function 11b detects the presence or absence of microorganisms in each droplet on the basis of whether fluorescence is detected. Furthermore, on the basis of the wavelength (or spectrum) and intensity of the fluorescence, the detection function 11b detects the type and number of microorganisms encapsulated in each droplet.

The sorting function 11c according to the second embodiment sorts droplets on the basis of the detection result by the detection function 11b. Specifically, the sorting function 11c sorts droplets into the droplets each encapsulating a predetermined number of (for example, one) microorganism for each type of microorganisms. For example, the sorting function 11c controls the sorting unit 16 so that droplets exhibiting a predetermined fluorescence intensity (for example, a fluorescence intensity generated when one microorganism is encapsulated) are collected for each wavelength (or spectrum) of fluorescence.

The microfluidic chip 20 according to the second embodiment has a plurality of collection ports 28, and a plurality of collection units 17 are connected to the collection ports 28, respectively. Furthermore, the sorting unit 16 according to the second embodiment is formed to be able to change the application of an electric field so that droplets for each type of microorganisms can be selectively moved in the direction to the corresponding collection port 28. Then, the sorting function 11c controls the sorting unit 16 so that the droplet for each type of microorganisms are collected by the different collection unit 17.

As described above, according to the second embodiment, a plurality of droplets are sorted separately according to the fluorescence detection result. Consequently, the droplet collection unit 1 according to the second embodiment can detect and sort multiple types of microorganisms at the same time.

### Other Embodiments

Although the first and second embodiments have been described so far, various different embodiments may be carried out in addition to the first and second embodiments described above.

In the embodiments described above, the case of sorting droplets each encapsulating one microorganism (virus) has been described. However, embodiments are not limited thereto, and may include a case where droplets each encapsulating microorganisms the number of which is two or more that is desired are sorted. In such a case, the concentration of microorganisms in the second fluid is adjusted. Then, on the basis of the detection result by the detection function 11b, the sorting function 11c controls the sorting unit 16 so that droplets exhibiting fluorescence intensity corresponding to the two or more desired numbers are flowed toward the collection port 28.

Furthermore, the droplet collection unit 1 can also sort droplets by the number of microorganisms encapsulated in each droplet. That is, on the basis of determination results, the sorting function 11c sorts the droplets by the number of microorganisms contained in each droplet. In such a case, as in the second embodiment, the microfluidic chip 20 includes a plurality of the collection ports 28 and the plurality of collection units 17 are connected to the collection ports 28, respectively. The sorting unit 16 is formed to change the application of an electric field so that droplets for each type of microorganisms can be selectively moved in the direction to a corresponding collection port 28. Then, on the basis of the difference in fluorescence intensity, the sorting function 11c controls the sorting unit 16 so that droplets each having the number of encapsulated microorganisms different from one another are collected by the different collection units 17.

Furthermore, in the embodiments described above, the case of detecting fluorescence based on a fluorescent substance produced by an enzyme reaction has been described. However, embodiments are not limited thereto, and may also include a case of detecting light emission based on a fluorescent substance produced by an enzyme reaction.

Furthermore, in the embodiments described above, the case of sorting droplets by dielectrophoresis has been described. However, embodiments are not limited thereto, and any method capable of sorting droplets may be used.

Furthermore, in the embodiments described above, an example of the droplet collection unit formed to be joinable to the system using the collected droplets has been described. However, embodiments are not limited thereto, and the droplet collection method described above may also be performed by a droplet collection apparatus not formed to be joinable to the system using the collected droplets. Furthermore, the droplet collection apparatus may include, for example, a configuration using the collected droplets, in addition to a configuration for performing the droplet collection method described above. For example, the droplet collection apparatus may be configured to perform various analyses and tests using microorganisms encapsulated in a droplet. In such a case, processing circuitry included in the droplet collection apparatus performs a processing function of performing processing on droplets sorted by the sorting function 11c, in addition to the control function 11a, the detection function 11b, and the sorting function 11c described above. That is, the processing function controls analyses and tests using microorganisms encapsulated in the sorted droplets. The processing function is an example of a processing unit.

In FIG. 1, an example of the case where each processing function is implemented by a single processing circuitry 11 has been described; however, embodiments are not limited thereto. For example, the processing circuitry 11 may be configured by combining a plurality of independent processors, and respective processors may implement respective processing functions by executing respective computer programs. Respective processing functions of the processing circuitry 11 may be implemented by being appropriately distributed or integrated into a single or a plurality of processing circuits.

Furthermore, the term “processor” used in the description of each of the embodiments described above means, for example, a circuit such as a central processing unit (CPU), a graphics processing unit (GPU), an application specific integrated circuit (ASIC), or a programmable logic device (for example, a simple programmable logic device (SPLD)), a complex programmable logic device (CPLD), and a field programmable gate array (FPGA)). Instead of storing the computer programs in a memory, the computer programs may be directly incorporated in the circuit of the processor. In such a case, the processor implements the functions by reading and executing the computer programs incorporated in the circuit. Furthermore, each processor of the present embodiment is not limited to being configured as a single circuit for each processor, and one processor may be configured by combining a plurality of independent circuits to implement the functions thereof.

According to at least one of the embodiments described above, it is possible to collect a droplet in which the amount of microorganisms encapsulated in the droplet is adjusted.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A droplet collection unit comprising:
a generator configured to produce droplets each containing a microorganism and a substrate that reacts with an enzyme derived from the microorganism; and
processing circuitry configured to
detect a reaction between the enzyme and the substrate in each droplet, and
sort the droplets on the basis of a detection result of the reaction.

2. The droplet collection unit according to claim 1, wherein the processing circuitry is configured to
determine an amount of the microorganisms contained in each droplet on the basis of the detection result of the reaction, and
sort the droplets on the basis of a result of the determination.

3. The droplet collection unit according to claim 2, wherein the processing circuitry is configured to
determine the number of the microorganisms contained in each droplet on the basis of the detection result of the reaction, and
sort the droplets on the basis of a result of the determination.

4. The droplet collection unit according to claim 1, wherein the processing circuitry is configured to detect fluorescence based on the reaction between the enzyme and the substrate.

5. The droplet collection unit according to claim 3, wherein the processing circuitry is configured to
determine the number of the microorganisms contained in each droplet on the basis of an intensity of fluorescence based on the reaction between the enzyme and the substrate, and
sort the droplets on the basis of a result of the determination.

6. The droplet collection unit according to claim 5, wherein the processing circuitry is configured to sort the droplets by the number of the microorganisms contained in each droplet on the basis of the result of the determination.

7. The droplet collection unit according to claim 1, wherein the processing circuitry is configured to sort, by dielectrophoresis, the droplets produced in a microchannel.

8. The droplet collection unit according to claim 1, wherein the processing circuitry is configured to adjust a temperature of the droplets.

9. A droplet collection apparatus comprising:
a generator configured to produce droplets each containing a microorganism and a substrate that reacts with an enzyme derived from the microorganism; and
processing circuitry configured to
detect a reaction between the enzyme and the substrate in each droplet,
sort the droplets on the basis of a detection result of the reaction, and
perform processing on the sorted droplets.

10. A droplet collection method comprising:
producing droplets each containing a microorganism and a substrate that reacts with an enzyme derived from the microorganism;
detecting a reaction between the enzyme and the substrate in each droplet; and
sorting the droplets on the basis of a detection result of the reaction.

11. The droplet collection method according to claim 10, wherein an amount of the microorganisms contained in each droplet is determined on the basis of the detection result of the reaction, and the droplets are sorted on the basis of a result of the determination.

12. The droplet collection method according to claim 11, wherein the number of the microorganisms contained in each droplet is determined on the basis of the detection result of the reaction, and the droplets are sorted on the basis of a result of the determination.

13. The droplet collection method according to claim 10, wherein fluorescence based on the reaction between the enzyme and the substrate is detected.

14. The droplet collection method according to claim 12, wherein the number of the microorganisms contained in each droplet is determined on the basis of an intensity of fluorescence based on the reaction between the enzyme and the substrate, and the droplets are sorted on the basis of a result of the determination.

15. The droplet collection method according to claim 14, wherein the droplets are sorted by the number of the microorganisms contained in each droplet on the basis of the result of the determination.

16. The droplet collection method according to claim 10, wherein the droplets produced in a microchannel are sorted by dielectrophoresis.

17. The droplet collection method according to claim 10, further comprising: adjusting a temperature of the droplets.
